(19) United States
(12) Patent Application Publication
(10) Pub. No.: US 2004/0248849 A1
(43) Pub. Date: Dec. 9, 2004

(54) 3-Aryl-A-oxy substituted propanoic acids and a process for their preparation

(76) Inventors: Rajender Kumar Potlapally, Andra Pradesh (IN); Venkata Rama Murali Krishna Reddy Velagala, Andra Pradesh (IN); Ramabhadra Sarma Mamillapalli, Andra Pradesh (IN); Om Reddy Gaddam, Andra Pradesh (IN)

Correspondence Address: Ladas & Parry, 26 West 61st Street, New York, NY, 10023, US

(21) Appl. No.: 10/481,735

(22) PCT Filed: Jun. 28, 2001
(86) PCT No.: PCT/IN01/00124

(51) Int. Cl. C07C 69/76; A61K 31/695; A61K 31/235

(52) U.S. Cl. 514/63; 556/437; 560/60; 514/543

(57) ABSTRACT

The present invention relates to novel antidiabetic compounds, their derivatives, their analogs, their tautomeric forms, their stereoisomers, their polymorphs and pharmaceutically acceptable compositions containing them. More particularly, the present invention relates to novel 3-aryl-α-oxy substituted propanoic acids of the general formula (I), their derivatives, their analogs, their tautomeric forms, their stereoisomers, their polymorphs and pharmaceutically acceptable compositions containing them. Formula (I) where R¹ represents t-butyldimethyl silyl, trimethyl silyl or alkoxyalkyl group; R² represents hydrogen or substituted or unsubstituted (C₁-C₆)alkyl group.

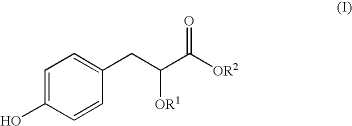

### FIELD OF THE INVENTION

The present invention relates to novel antidiabetic compounds, their derivatives, their analogs, their tautomeric forms, their stereoisomers, their polymorphs and pharmaceutically acceptable compositions containing them.

More particularly, the present invention relates to novel 3-aryl-α-oxy substituted propanoic acids of the general formula (I), their derivatives, their analogs, their tautomeric forms, their stereoisomers, their polymorphs and pharmaceutically acceptable compositions containing them.

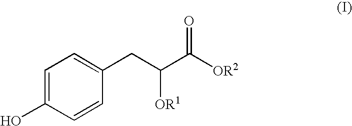

where R¹ represents t-butyldimethyl silyl, trimethyl silyl or alkoxyalkyl group; R² represents hydrogen or substituted or unsubstituted (C₁-C₆)alkyl group.

The present invention also relates to a process for the preparation of compounds of formula (1).

The present invention also relates to novel intermediate of formula (VI) and its use in the preparation of compounds of formula (I).

The compounds of formula (I) are useful in lowering the plasma glucose, triglyceride, total cholesterol (TC); increase high density lipoprotein (HDL) and decrease low density lipoprotein (LDL).

The compounds of formula (I) are useful in reducing body weight, glucose intolerance and for the treatment and/or prophylaxis of diseases such as hypertension, coronary heart disease, atherosclerosis, stroke, peripheral vascular diseases and related disorders. The compound of formula (I) is also useful for the treatment and/or prophylaxis of insulin resistance (type II diabetes).

The compounds of formula (1) are also useful as intermediates for the preparation of many pharmaceutically active compounds. Few representative examples of such compounds are

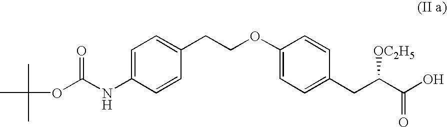

disclosed in WO 99/62870 and

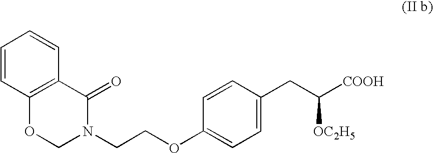

disclosed in WO 99/16758. The compounds of formulae (IIa) and (IIb) are shown to have potent blood glucose lowering, triglyceride lowering, cholesterol lowering and body weight reducing activities.

### BACKGROUND OF INVENTION

Diabetes and insulin resistance is yet another disease which severely effects the quality of life of a large population in the world. Insulin resistance is the diminished ability of insulin to exert its biological action across a broad range of concentrations. In insulin resistance, the body secretes abnormally high amounts of insulin to compensate for this defect; failing which, the plasma glucose concentration inevitably rises and develops into diabetes. Among the developed countries, diabetes mellitus is a common problem and is associated with a variety of abnormalities including obesity, hypertension, hyperlipidemia (J. Clin. Invest., (1985) 75: 809-817; N. Engl. J. Med. (1987) 317: 350-357; J. Clin. Endocrinol. Metab., (1988) 66: 580-583; J. Clin. Invest, (1975) 68: 957-969) and other renal complications (See Patent Application No. WO 95/21608). It is now increasingly being recognized that insulin resistance and relative hyperinsulinemia have a contributory role in obesity, hypertension, atherosclerosis and type 2 diabetes mellitus. The association of insulin resistance with obesity, hypertension and angina has been described as a syndrome having insulin resistance as the central pathogenic link-Syndrome-X.

Cernerud et. al., in Tetrahedron Asymmetry, 7(10), 2863-2870, 1996, disclosed di-t-butyl dimethyl silyloxy benzenepropionic acid of the formula (III)

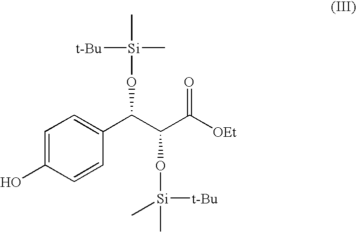

### OBJECTIVE OF PRESENT INVENTION

The main objective of the present invention is to provide novel compounds of the formula (I) for the treatment and/or prophylaxis of diabetes with high chiral purity, which can be used in the synthesis of pharmaceutically acceptable compounds, which will not have problems of racemization in subsequent steps, when used in the preparation of pharmaceutically acceptable compounds.

Another objective of the present invention is to provide a simple and robust process for the preparation of the compound of formula (I).

### DETAILED DESCRIPTION OF THE INVENTION

Accordingly, the present invention provides novel 3-aryl-α-oxy substituted propanoic acid and their derivatives, their stereoisomers, their polymorphs having the formula (I)

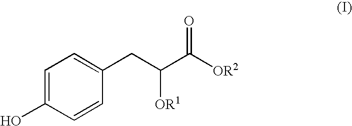

where R¹ represents t-butyldimethyl silyl, trimethyl silyl or alkoxyalkyl group;

R² represents hydrogen or substituted or unsubstituted (C₁-C₆)alkyl group.

The term alkoxyalkyl represents methoxymethyl, methoxyethyl, ethoxymethyl, ethoxyethyl and the like.

The term (C₁-C₆)alkyl group represents groups such as methyl, ethyl, propyl, isopropyl, t-butyl, n-butyl and the like.

Suitable substituents on the alkyl group represented by R² may be selected from hydroxy or alkoxy group such as methoxy, ethoxy, propoxy and the like.

Particularly useful compounds of the formula (I) according to the present invention, include:

(±) 3-(4-Hydroxyphenyl)-2-(t-butyl dimethyl silyloxy)propanoic acid;

(+) 3-(4-Hydroxyphenyl)-2-(t-butyl dimethyl silyloxy)propanoic acid;

(−) 3-(4-Hydroxyphenyl)-2-(t-butyl dimethyl silyloxy)propanoic acid;

(±) Methyl 3-(4-hydroxyphenyl)-2-(t-butyl dimethyl silyloxy)propanoate;

(+) Methyl 3-(4-hydroxyphenyl)-2-(t-butyl dimethyl silyloxy)propanoate;

(−) Methyl 3-(4-hydroxyphenyl)-2-(t-butyl dimethyl silyloxy)propanoate;

(±) Ethyl 3-(4-hydroxyphenyl)-2-(t-butyl dimethyl silyloxy)propanoate;

(+) Ethyl 3-(4-hydroxyphenyl)-2-(t-butyl dimethyl silyloxy)propanoate;

(−) Ethyl 3-(4-hydroxyphenyl)-2-(t-butyl dimethyl silyloxy)propanoate;

(±) Isopropyl 3-(4-hydroxyphenyl)-2-(t-butyl dimethyl silyloxy)propanoate;

(+) Isopropyl 3-(4-hydroxyphenyl)-2-(t-butyl dimethyl silyloxy)propanoate;

(−) Isopropyl 3-(4-hydroxyphenyl)-2-(t-butyl dimethyl silypoxy)propanoate;

(±) Isopropyl 3-(4-hydroxyphenyl)-2-methoxymethoxy propanoate;

(+) Isopropyl 3-(4-hydroxyphenyl)-2-methoxymethoxy propanoate;

(−) Isopropyl 3-(4-hydroxyphenyl)-2-methoxymethoxy propanoate;

(±) Isopropyl 3-(4-hydroxyphenyl)-2-methoxymethoxy propanoate;

(+) Isopropyl 3-(4-hydroxyphenyl)-2-methoxymethoxy propanoate;

(−) Isopropyl 3-(4-hydroxyphenyl)-2-methoxymethoxy propanoate;

(±) 3-(4-Hydroxyphenyl)-2-(trimethyl silyloxy)propanoic acid;

(+) 3-(4-Hydroxyphenyl)-2-(trimethyl silyloxy)propanoic acid;

(−) 3-(4-Hydroxyphenyl)-2-(trimethyl silyloxy)propanoic acid;

(±) Methyl 3-(4-hydroxyphenyl)-2-(trimethyl silyloxy)propanoate;

(+) Methyl 3-(4-hydroxyphenyl)-2-(trimethyl silyloxy)propanoate;

(−) Methyl 3-(4-hydroxyphenyl)-2-(trimethyl silyloxy)propanoate;

(±) Ethyl 3-(4-hydroxyphenyl)-2-(trimethyl silyloxy)propanoate;

(+) Ethyl 3-(4-hydroxyphenyl)-2-(trimethyl silyloxy)propanoate;

(−) Ethyl 3-(4-hydroxyphenyl)-2-(trimethyl silyloxy)propanoate;

(±) Isopropyl 3-(4-hydroxyphenyl)-2-(trimethyl silyloxy)propanoate;

(+) Isopropyl 3-(4-hydroxyphenyl)-2-(trimethyl silyloxy)propanoate;

(−) Isopropyl 3-(4-hydroxyphenyl)-2-(trimethyl silyloxy)propanoate;

According to another embodiment of the present invention there is provided a process for the preparation of novel 3-aryl-α-oxy substituted propanoic acid and their derivatives, having the formula (I)

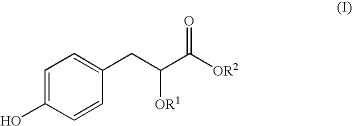

where R¹ represents t-butyldimethyl silyl, trimethyl silyl or alkoxyalkyl group;

R² represents hydrogen or substituted or unsubstituted (C₁-C₆)alkyl group, which comprises:

(i). esterifying the compound of formula (IV) where R³ represents benzyl using alkylating agent to produce compound of formula (V) where R² represents (C₁-C₆)alkyl group,

ii). protecting the compound of formula (V) with a protecting agent in the presence of a base and a solvent to obtain compound of formula (VI) where R² represents (C₁-C₆)alkyl group and R¹ and R³ are as defined above and

iii). debenzylating the compound of formula (VI) where R³ represents benzyl using aqueous alcohol in the presence of metal catalysts to yield pure compound of formula (1) where R¹ and R² are as defined above.

The process explained above is shown in scheme-1 below:

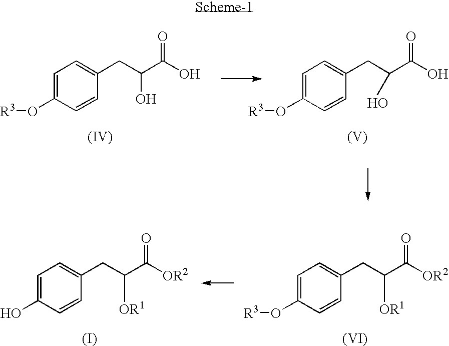

The esterification of compound of formula (I) to obtain compound of formula (V) may be carried out using alcohol such as methanol, ethanol, propanol, isopropanol and the like under acidic conditions in the presence of sulfuric acid, methane sulfonic acid, thionyl chloride, p-TSA, amberlite resin or HCl or the reaction may be carried out using ethyl iodide, DES, DMS and the like under basic conditions in the presence of sodium carbonate, potassium carbonate, sodium methoxide and the like. The reaction may be carried out 30° C. to reflux temperature of the solvent used. The duration of the reaction may range from 2 to 20 h.

The protection of compound of formula (V) may be carried out with protecting agent such as t-butyldirnethyl silyl chloride, trimethyl silyl chloride, alkoxyalcohols such as methoxymethanol, ethoxymethanol and the like in the presence of bases such as imidazole, triethyl amine, potassium carbonate and the like. The reaction may be carried out in the presence of solvents such as toluene, DMF, DCE, DCM, diethyl acetamide, N-methyl pyrrolidone, ethyl acetate, acetonitrile and the like. The reaction may be carried out at a temperature in the range of 10 to 90° C. and the duration of the reaction may range from 2-30 h.

The debenzylation of the compound of formula (VI) to yield compound of formula (I) may be carried out using THF, aqueous acetic acid, ethyl acetate, aqueous (C₁-C₆) alcohols such as aqueous methanol, ethanol, propanol, isopropanol and the like in the presence of metal catalysts such as Pd/C.

According to another embodiment of the present invention there is provided a novel intermediate of formula (VI)

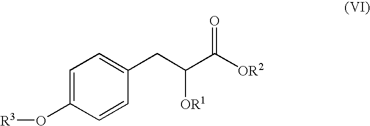

where R¹ represents t-butyldimethyl silyl, trimethyl silyl or alkoxyalkyl group;

R² represents hydrogen or substituted or unsubstituted (C₁-C₆)akyl group, R³ represents benzyl.

The compounds of formula (I) are useful in the preparation of pharmaceutically important compounds such as

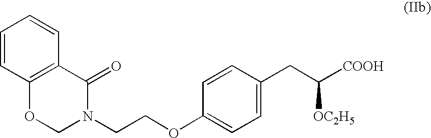

The process for preparing the compounds of formula (IIb) starting from compound of formula (I) is as shown in scheme-3:

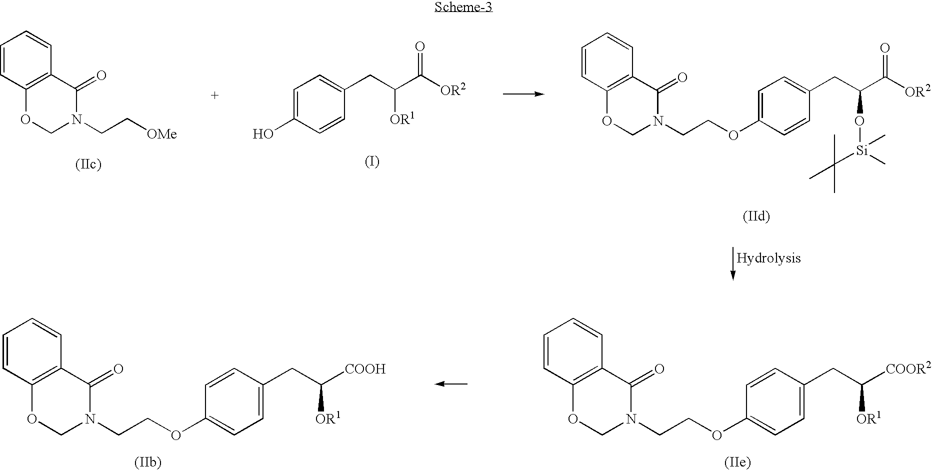

It is appreciated that in any of the above mentioned reactions, any reactive group in the substrate molecule may be protected according to conventional chemical practice. Suitable protecting groups in any of the above mentioned reactions are tertiarybutyl dimethyl silylchloride, methoxymethyl chloride and the like. The methods of formation and removal of such protecting groups are those conventional methods appropriate to the molecule being protected.

The stereoisomers of the compounds forming part of this invention may be prepared by using compound of formula (I) in its single enantiomeric form in the process by resolving the mixture of stereoisomers by conventional methods. Some of the preferred methods include use of microbial resolution, resolving the diastereomeric salts formed with optically pure bases such as brucine, cinchona alkaloids and their derivatives, optically pure 2-alkyl phenethyl amine, phenyl glycinol and the like. The diastereomeric salts may be obtained in pure form by fractional crystallization. Commonly used methods are compiled by Jaques et al in “Enantiomers, Racemates and Resolution” (Wiley Interscience, 1981).

Various polymorphs of compound of general formula (I) forming part of this invention may be prepared by crystallization of compound of formula (I) under different conditions. For example, using different solvents commonly used or their mixtures for recrystallization; crystallizations at different temperatures; various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffraction or such other techniques.

The invention is described in the examples given below which are provided by way of illustration only and therefore should not construed to limit the scope of the invention.

### EXAMPLE 1

### Step (i)

### Preparation of methyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g), methanol (30 ml) and sulfuric acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 85-95%.

### Step (ii)

### Preparation of methyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser methyl 2(S)-hydroxy-3-(4-benzyloxyphenyl)propanoate (2.5 g), obtained in step (i), N-methyl pyrrolidone (12.5 ml), triethyl amine (2.20 g) to and tertiary butyl dimethyl silyl chloride (2.62 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil, yield 82-85%.

### Step (iii)

### Preparation of methyl 2(S)-tertiary butyl dimethyl silyloxy-3-(4-hydroxyphenyl)propanoate (I)

In a 250 ml parr hydrogenation flask, palladium carbon (5%, 0.3 g) was taken. Methyl 2(S)-tertiary butyl dimethyl silyloxy-3-(4-benzyloxyphenyl)propanoate (3 g) dissolved in methanol (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 90-95%.

### EXAMPLE 2

### Step (i)

### Preparation of ethyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) ethanol (30 ml) and sulfuric acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonrate solution. The organic layer was separated and the aqueous layer was extracted with ethyl acetate (30 ml). The combined organic layers were washed with water (30 ml) and concentrated on a rotavapour under reduced pressure to yield the title compound, yield 80-90%.

### Step (ii)

### Preparation of ethyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser ethyl 2(S)-hydroxy-3-(4-benzyloxyphenyl)propanoate (2.5 g), obtained in step (i), DMF (12.5 ml), imidazole (1.41 g) and tertiary butyl dimethyl silyl chloride (2.49 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil.

The crude compound was distilled at reduced pressure (2 mm/Hg) and 200-220° C. (vapour temp) to obtain the pure title compound as a pale yellow liquid, yield 82-85%.

### Step (iii)

### Preparation of ethyl 2(S)-tertiary butyl dimethyl silyloxy-3-(4-hydroxyphenyl)propanoate (I)

In a 250 ml parr hydrogenation flask, palladium carbon (5%, 0.3 g) was taken. Ethyl 2(S)-tertiary butyl dimethyl silyloxy-3-(4-benzyloxyphenyl)propanoate (3 g) dissolved in ethanol (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 92-96%.

### EXAMPLE 3

### Step (i)

### Preparation of propyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) propanol (30 ml) and sulfuric acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 91-95%.

### Step (ii)

### Preparation of propyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser propyl 2(S)-hydroxy-3-(4-benzyloxyphenyl)propanoate (2.5 g), obtained in step (i), DMF (12.5 ml), triethyl amine (2.01 g) and tertiary butyl dimethyl silyl chloride (2.38 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil.

The crude compound was distilled at reduced pressure (2 mm/Hg) and 200-220° C. (vapour temp) to obtain the pure title compound as a pale yellow liquid, yield 85-90%.

### Step (iii)

### Preparation of propyl 2(S)-tertiary butyl dimethyl silyloxy-3-(4-hydroxyphenyl)propanoate (I)

In a 250 ml parr hydrogenation flask, palladium carbon (5%, 0.3 g) was taken. Propyl 2(S)-tertiary butyl dimethyl silyloxy-3-(4-benzyloxyphenyl)propanoate (3 g) dissolved in propanol (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 85-88%.

### EXAMPLE 4

### Step (i)

### Preparation of isopropyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) isopropanol (30 ml) and sulfuric acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 90-97%.

### Step (ii)

### Preparation of isopropyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser isopropyl 2(S)-hydroxy-3-(4-benzyloxyphenyl)propanoate (2.5 g), obtained in step (i), N-methyl pyrrolidone (12.5 ml), triethyl amine (2.01 g) and tertiary butyl dimethyl silyl chloride (2.38 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil, yield 90-95%.

### Step (iii)

### Preparation of isopropyl 2(S)-tertiary butyl dimethyl silyloxy-3-(4-hydroxyphenyl)propanoate (I)

In a 250 ml parr hydrogenation flask, palladium carbon (5%, 0.3 g) was taken. Isopropyl 2(S)-tertiary butyl dimethyl silyloxy-3-(4-benzyloxyphenyl)propanoate (3 g) dissolved in THF (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 80-85%.

### EXAMPLE 5

### Step (i)

### Preparation of methyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) methanol (30 ml) and methane sulfonic acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 87-93%.

### Step (ii)

### Preparation of methyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser methyl 2(S)-hydroxy-3-(4-benzyloxyphenyl)propanoate (2.5 g), obtained in step (i), DMF (12.5 ml), imidazole (1.48 g) and tertiary butyl dimethyl silyl chloride (2.62 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil, yield 79-85%.

### Step (iii)

### Preparation of methyl 2(S)-tertiary butyl dimethyl silyloxy-3-(4-hydroxyphenyl)propanoate (I)

In a 250 ml parr hydrogenation flask, palladium carbon (5%, 0.3 g) was taken. Methyl 2(S)-tertiary butyl dimethyl silyloxy-3-(4-benzyloxyphenyl)propanoate (3 g) dissolved in methanol (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 90-95%.

### EXAMPLE 6

### Step (i)

### Preparation of ethyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) ethanol (30 ml) and methane sulfonic acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was, extracted with ethyl acetate (30 ml). The combined organic layers were washed with water (30 ml) and concentrated on a rotavapour under reduced pressure to yield the title compound, yield 84-88%.

### Step (ii)

### Preparation of ethyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser ethyl 2(S)-hydroxy-3-(4-benzyloxyphenyl)propanoate (2.5 g), obtained in step (i), N-methyl pyrrolidone (12.5 ml), triethyl amine (2.10 g) and tertiary butyl dimethyl silyl chloride (2.49 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil, yield 81-86%.

### Step (iii)

### Preparation of ethyl 2(S)-tertiary butyl dimethyl silyloxy-3-(4-hydroxyphenyl)propanoate (I)

In a 250 ml parr hydrogenation flask, palladium carbon (5%, 0.3 g) was taken. Ethyl 2(S)-tertiary butyl dimethyl silyloxy-3-(4-benzyloxyphenyl)propanoate (3 g) dissolved in methanol (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 90-92%.

### EXAMPLE 7

### Step (i)

### Preparation of propyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) propanol (30 ml) and methane sulfonic acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 86-88%.

### Step (ii)

### Preparation of propyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser propyl 2(S)-hydroxy-3-(4-benzyloxyphenyl)propanoate (2.5 g), obtained in step (i), N-methyl pyrrolidone (12.5 ml), imidazole (1.35 g) and tertiary butyl dimethyl silyl chloride (2.38 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil.

The crude compound was distilled at reduced pressure (2 mm/Hg) and 200-220° C. (vapour temp) to obtain the, pure title compound as a pale yellow liquid, yield 85-88%.

### Step (iii)

### Preparation of propyl 2(S)-tertiary butyl dimethyl silyloxy-3-(4-hydroxyphenyl)propanoate (I)

In a 250 ml parr hydrogenation flask, palladium carbon (5%, 0.3 g) was taken. Propyl 2(S)-tertiary butyl dimethyl silyloxy-3-(4-benzyloxyphenyl)propanoate (3 g) dissolved in methanol (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 88-90%.

### EXAMPLE 8

### Step (i)

### Preparation of isopropyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) isopropanol (30 ml) and methane sulfonic acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 88-92%.

### Step (ii)

### Preparation of isopropyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser isopropyl 2(S)-hydroxy-3-(4-benzyloxyphenyl)propanoate (2.5 g), obtained in step (i), DMF (12.5 ml), imidazole (1.35 g) and tertiary butyl dimethyl silyl chloride (2.38 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil, yield 88-92%.

### Step (iii)

### Preparation of isopropyl 2(S)-tertiary butyl dimethyl silyloxy-3-(4-hydroxyphenyl)propanoate (I)

In a 250 ml parr hydrogenation flask, palladium carbon (5%, 0.3 g) was taken. Isopropyl 2(S)-tertiary butyl dimethyl silyloxy-3-(4-benzyloxyphenyl)propanoate (3 g) dissolved in isopropanol (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 90-96%.

### EXAMPLE 9

### Step (i)

### Preparation of methyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) methanol (30 ml) and thionyl chloride (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 88-90%.

### Step (ii)

### Preparation of methyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser methyl 2(S)-hydroxy-3-(4-benzyloxyphenyl)propanoate (2.5 g), obtained in step (i), diethyl acetamide (12.5 ml), potassium carbonate (3.01 g) and tertiary butyl dimethyl silyl chloride (2.62 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil, yield 65-70%.

### Step (iii)

### Preparation of methyl 2(S)-tertiary butyl dimethyl silyloxy-3-(4-hydroxyphenyl)propanoate (I)

In a 250 ml parr hydrogenation flask, palladium carbon (5%, 0.3 g) was taken. Methyl 2(S)-tertiary butyl dimethyl silyloxy-3-(4-benzyloxyphenyl)propanoate (3 g) dissolved in isopropanol (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 92-95%.

### EXAMPLE 10

### Step (i)

### Preparation of ethyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) ethanol (30 ml) and amberlite resin (1.5 g) were taken and refluxed for 16 h. The progress of the reaction was monitored by TLC. Refluxing was continued, till the starting material has disappeared on TLC. The reaction mass was cooled to room temperature and filtered the resin and transferred the filtrate into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The organic layers were washed with water (30 ml) and concentrated on a rotavapour under reduced pressure to yield the title compound, yield 70-75%.

### Step (ii)

### Preparation of ethyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser ethyl 2(S)-hydroxy-3-(4-benzyloxyphenyl)propanoate (2.5 g), obtained in step (i), dimethyl acetamide (12.5 ml), potassium carbonate (2.87 g) and tertiary butyl dimethyl silyl chloride (2.49 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil, yield 80-88%.

### Step (iii)

### Preparation of ethyl 2(S)-tertiary butyl dimethyl silyloxy-3-(4-hydroxyphenyl)propanoate (I)

In a 250 ml parr hydrogenation flask, palladium carbon (5%, 0.3 g) was taken. Ethyl 2(S)-tertiary butyl dimethyl silyloxy-3-(4-benzyloxyphenyl)propanoate (3 g) dissolved in THF (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 75-88%.

### EXAMPLE 11

### Step (i)

### Preparation of isopropyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) isopropanol (30 ml) and amberlite resin (1.5 g) were taken and refluxed for 16 h. The progress of the reaction was monitored by TLC. Refluxing was continued, till the starting material has disappeared on TLC. The reaction mass was cooled to room temperature and filtered the resin and transferred the filtrate into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 80-84%.

### Step (ii)

### Preparation of isopropyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser isopropyl 2(S)-hydroxy-3-(4-benzyloxyphenyl)propanoate (2.5 g), obtained in step (i), diethyl acetamide (12.5 ml), potassium carbonate (2.74 g) and tertiary butyl dimethyl silyl chloride (2.38 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil.

The crude compound was distilled at reduced pressure (2 mm/Hg) and 200-220° C. (vapour temp) to obtain the pure title compound as a pale yellow liquid, yield 92-95%.

### Step (iii)

### Preparation of isopropyl 2(S)-tertiary butyl dimethyl silyloxy-3-(4-hydroxyphenyl)propanoate (I)

In a 250 ml parr hydrogenation flask, palladium carbon (5%, 0.3 g) was taken. Isopropyl 2(S)-tertiary butyl dimethyl silyloxy-3-(4-benzyloxyphenyl)propanoate (3 g) dissolved in acetone (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 70-75%.

### EXAMPLE 12

### Step (i)

### Preparation of isopropyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) isopropanol (30 ml) and thionyl chloride (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 88-89%.

### Step (ii)

### Preparation of isopropyl 2(S)-tertiary butyl dimethyl silyloxy-3-(4-benzyloxyphenyl)propanoate (VI)

In a 50 ml 3 neck round bottom flask, fitted with a mechanical stirrer and reflux condenser isopropyl 2(S)-hydroxy-3-(4-benzyloxyphenyl)propanoate (2.5 g), obtained in step (i), diethyl acetamide (12.5 ml), potassium carbonate (2.74 g) and tertiary butyl dimethyl silyl chloride (2.38 g) were taken. The reaction mass was heated to 60-70° C. and maintained at this temperature for a period of 4 h. The progress of the reaction was monitored by TLC. After completion of the reaction, the reaction mixture was cooled to room temperature, quenched with 5% aqueous NaHCO₃ (25 ml) and extracted with ethyl acetate (3×25 ml). The organic layer was washed with water and concentrated to yield crude title compound as brown coloured oil, yield 92-95%.

### Step (iii)

### Preparation of isopropyl 2(S)-tertiary butyl dimethyl silyloxy-3-(4-hydroxyphenyl)propanoate (I)

In a 250 ml parr hydrogenation flask, palladium carbon (5%, 0.3 g) was taken. Isopropyl 2(S)-tertiary butyl dimethyl silyloxy-3-(4-benzyloxyphenyl)propanoate (3 g) dissolved in methanol (50 ml) was added and fixed to a parr hydrogenation apparatus. The reaction mass was hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 92-95%.

### EXAMPLE 13

### Step (i)

### Preparation of methyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) methanol (30 ml) and sulfuric acid (6.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 85-95%.

### Step (ii)

### Preparation of methyl 2(S)-trimethyl silyloxy-3-(4-benzyloxyphenyl)propanoate (VI)

In a 100 ml round bottom flask methyl 2(S)-hydroxy-3-(4-benzyloxyphenyl)propanoate (2.0 g), DMF (20 ml), imidazole (1.89 g) were taken. Trimethyl silyl chloride (3.77 g, 0.0349 M) was added slowly. The reaction mass was heated to 60-70° C. and maintained at this temperature for a period of 20-24 h. The progress of the reaction was monitored by TLC. After completion of the reaction, the reaction mixture was cooled to room temperature, quenched with 5% aqueous sodium bicarbonate and extracted with ethyl acetate. The organic layer was washed with water and concentrated to yield crude title compound as brown coloured oil.

### Step (iii).

### Preparation of methyl 2(S)-trimethyl silyloxy-3-(4-hydroxyphenyl)propanoate (I)

In 250 ml parr hydrogenation flask, palladium carbon (5%, 1 g) slurred in water (1 ml) was taken. Methyl 2(S)-trimethyl silyloxy-3-(4-benzyloxyphenyl)propanoate (3 g) dissolved in methanol (100 ml) was added and hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 80-82%.

### EXAMPLE 14

### Preparation of methyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) methanol (30 ml) and methane sulfonic acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 87-93%.

### Step (ii)

### Preparation of methyl 2(S)-trimethyl silyloxy-3-(4-benzyloxyphenyl)propanoate (VI)

In a 100 ml round bottom flask methyl 2(S)-hydroxy-3-(4-benzyloxyphenyl)propanoate (2.0 g), N-methyl pyrrolidone (20 ml), triethyl amine (1.76 g) were taken. Trimethyl silyl chloride (3.77 g, 0.0349 M) was added slowly. The reaction mass was heated to 60-70° C. and maintained at this temperature for a period of 20-24 h. The progress of the reaction was monitored by TLC. After completion of the reaction, the reaction mixture was cooled to room temperature, quenched with 5% aqueous sodium bicarbonate and extracted with ethyl acetate. The organic layer was washed with water and concentrated to yield crude title compound as brown coloured oil.

### Step (iii)

### Preparation of methyl 2(S)-trimethyl silyloxy-3-(4-hydroxyphenyl)propanoate (I)

In 250 ml parr hydrogenation flask, palladium carbon (5%, 1 g) slurred in water (1 ml) was taken. Methyl 2(S)-trimethyl silyloxy-3-(4-benzyloxyphenyl)propanoate (3 g) dissolved in methanol (100 ml) was added and hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 80-82%.

### EXAMPLE 15

### Step (i)

### Preparation of methyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) methanol (30 ml) and thionyl chloride (0.3 ml) were taken and refluxed for 16 h. The progress of the reaction was monitored by TLC. Refluxing was continued, till the string material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 88-90%.

### Step (ii)

### Preparation of methyl 2(S)-trimethyl silyloxy-3-(4-benzyloxyphenyl)propanoate (VI)

In a 100 ml round bottom flask methyl 2(S)-hydroxy-3-(4-benzyloxyphenyl)propanoate (2.0 g), diethyl acetamide (20 ml), potassium carbonate (2.41 g) were taken. Trimethyl silyl chloride (3.77 g, 0.0349 M) was added slowly. The reaction mass was heated to 60-70° C. and maintained at this temperature for a period of 20-24 h. The progress of the reaction was monitored by TLC. After completion of the reaction, the reaction mixture was cooled to room temperature, quenched with 5% aqueous sodium bicarbonate and extracted with ethyl acetate. The organic layer was washed with water and concentrated to yield crude title compound as brown coloured oil.

### Step (iii)

### Preparation of methyl 2(S)-trimethyl silyloxy-3-(4-hydroxyphenyl)propanoate (I)

In 250 ml parr hydrogenation flask, palladium carbon (5%, 1 g) slurred in water (1 ml) was taken. Methyl 2(S)-trimethyl silyloxy-3-(4-benzyloxyphenyl)propanoate (3 g) dissolved in isopropanol (100 ml) was added and hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 78-84%.

### EXAMPLE 16

### Step (i)

### Preparation of ethyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) ethanol (30 ml) and sulfuric acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 80-90%.

### Step (ii)

### Preparation of ethyl 2(S)-trimethyl silyloxy-3-(4-benzyloxyphenyl)propanoate (VI)

In a 100 ml round bottom flask ethyl 2(S)-hydroxy-3-(4-benzyloxyphenyl)propanoate (2.0 g), DMF (20 ml), imidazole (1.11 g) were taken. Trimethyl silyl chloride (3.59 g, 0.0332 M) was added slowly. The reaction mass was heated to 60-70° C. and maintained at this temperature for a period of 20-24 h. The progress of the reaction was monitored by TLC. After completion of the reaction, the reaction mixture was cooled to room temperature, quenched with 5% aqueous sodium bicarbonate and extracted with ethyl acetate. The organic layer was washed with water and concentrated to yield crude title compound as brown coloured oil.

### Step (iii)

### Preparation of ethyl 2(S)-trimethyl silyloxy-3-(4-hydroxyphenyl)propanoate (I)

In 250 ml parr hydrogenation flask, palladium carbon (5%, 1 g) slurred in water (1 ml) was taken. Ethyl 2(S)-trimethyl silyloxy-3-(4-benzyloxyphenyl)propanoate (3 g) dissolved in ethanol (100 ml) was added and hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 82-86%.

### EXAMPLE 17

### Step (i)

### Preparation of ethyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) ethanol (30 ml) and methane sulfonic acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 84-88%.

### Step (ii)

### Preparation of ethyl 2(S)-trimethyl silyloxy-3-(4-benzyloxyphenyl)propanoate (VI)

In a 100 ml round bottom flask ethyl 2(S)-hydroxy-3-(4-benzyloxyphenyl)propanoate (2.0 g), N-methyl pyrrolidone (20 ml), triethyl amine (1.68 g) were taken. Trimethyl silyl chloride (3.59 g, 0.0332 M) was added slowly. The reaction mass was heated to 60-70° C. and maintained at this temperature for a period of 20-24 h. The progress of the reaction was monitored by TLC. After completion of the reaction, the reaction mixture was cooled to room temperature, quenched with 5% aqueous sodium bicarbonate and extracted with ethyl acetate. The organic layer was washed with water and concentrated to yield crude title compound as brown coloured oil.

### Step (iii)

### Preparation of ethyl 2(S)-trimethyl silyloxy-3-(4-hydroxyphenyl)propanoate (I)

In 250 ml parr hydrogenation flask, palladium carbon (5%, 1 g) slurred in water (1 ml) was taken. Ethyl 2(S)-trimethyl silyloxy-3-(4-benzyloxyphenyl)propanoate (3 g) dissolved in methanol (100 ml) was added and hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 84-86%.

### EXAMPLE 18

### Step (i)

### Preparation of ethyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) ethanol (30 ml) and amberlite resin (1.5 g) were taken and refluxed for 16 h. The progress of the reaction was monitored by TLC. Refluxing was continued, till the starting material has disappeared on TLC. The reaction mass was cooled to room temperature and filtered the resin and transferred the filtrate into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 70-75%.

### Step (ii)

### Preparation of ethyl 2(S)-trimethyl silyloxy-3-(4-benzyloxyphenyl)propanoate (VI)

In a 100 ml round bottom flask ethyl 2(S)-hydroxy-3-(4-benzyloxyphenyl)propanoate (2.0 g), dimethyl acetamide (20 ml), potassium carbonate (2.29 g) were taken. Trimethyl silyl chloride (3.59 g, 0.0332 M) was added slowly. The reaction mass was heated to 60-70° C. and maintained at this temperature for a period of 20-24 h. The progress of the reaction was monitored by TLC. After completion of the reaction, the reaction mixture was cooled to room temperature, quenched with 5% aqueous sodium bicarbonate and extracted with ethyl acetate. The organic layer was washed with water and concentrated to yield crude title compound as brown coloured oil.

### Step (iii)

### Preparation of ethyl 2(S)-trimethyl silyloxy-3-(4-hydroxyphenyl)propanoate (I)

In 250 ml parr hydrogenation flask, palladium carbon (5%, 1 g) slurred in water (1 ml) was taken. Ethyl 2(S)-trimethyl silyloxy-3-(4-benzyloxyphenyl)propanoate (3 g) dissolved in TB:F (100 ml) was added and hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 79-81%.

### EXAMPLE 19

### Step (i)

### Preparation of propyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) propanol (30 ml) and sulfuric acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 91-95%.

### Step (ii)

### Preparation of propyl 2(S)-trimethyl silyloxy-3-(4-benzyloxyphenyl)propanoate (VI)

In a 100 ml round bottom flask propyl 2(S)-hydroxy-3-(4-benzyloxyphenyl)propanoate (2.0 g), DMF (20 ml), triethyl amine (1.60 g) were taken. Trimethyl silyl chloride (3.43 g, 0.0317 M) was added slowly. The reaction mass was heated to 60-70° C. and maintained at this temperature for a period of 20-24 h. The progress of the reaction was monitored by TLC. After completion of the reaction, the reaction mixture was cooled to room temperature, quenched with 5% aqueous sodium bicarbonate and extracted with ethyl acetate. The organic layer was washed with water and concentrated to yield crude title compound as brown coloured oil.

### Step (iii)

### Preparation of propyl 2(S)-trimethyl silyloxy-3-(4-hydroxyphenyl)propanoate (I)

In 250 ml parr hydrogenation flask, palladium carbon (5%, 1 g) slurred in water (1 ml) was taken. Propyl 2(S)-trimethyl silyloxy-3-(4-benzyloxyphenyl)propanoate (3 g) dissolved in propanol (100 ml) was added and hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 80-84%.

### EXAMPLE 20

### Step (i)

### Preparation of propyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) propanol (30 ml) and methane sulfonic acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 86-88%.

### Step (ii)

### Preparation of propyl 2(S)-trimethyl silyloxy-3-(4-benzyloxyphenyl)propanoate (VI)

In a 100 ml round bottom flask propyl 2(S)-hydroxy-3-(4-benzyloxyphenyl)propanoate (2.0 g), N-methyl pyrrolidone (20 ml), imidazole (1.08 g) were taken. Trimethyl silyl chloride (3.43 g, 0.0317 M) was added slowly. The reaction mass was heated to 60-70° C. and maintained at this temperature for a period of 20-24 h. The progress of the reaction was monitored by TLC. After completion of the reaction, the reaction mixture was cooled to room temperature, quenched with 5% aqueous sodium bicarbonate and extracted with ethyl acetate. The organic layer was washed with water and concentrated to yield crude title compound as brown coloured oil.

### Step (iii)

### Preparation of propyl 2(S)-trimethyl silyloxy-3-(4-hydroxyphenyl)propanoate (I)

In 250 ml parr hydrogenation flask, palladium carbon (5%, 1 g) slurred in water (1 ml) was taken. Propyl 2(S)-trimethyl silyloxy-3-(4-benzyloxyphenyl)propanoate (3 g) dissolved in methanol (100 ml) was added and hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 80-83%.

### EXAMPLE 21

### Step (i)

### Preparation of isopropyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) isopropanol (30 ml) and sulfuric acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 90-97%.

### Step (ii)

### Preparation of isopropyl 2(S)-trimethyl silyloxy-3-(4-benzyloxyphenyl)propanoate (VI)

In a 100 ml round bottom flask isopropyl 2(S)-hydroxy-3-(4-benzyloxyphenyl)propanoate (2.0 g), DMF (20 ml), imidazole (1.08 g) were taken. Trimethyl silyl chloride (3.43 g, 0.0317 M) was added slowly. The reaction mass was heated to 60-70° C. and maintained at this temperature for a period of 20-24 h. The progress of the reaction was monitored by TLC. After completion of the reaction, the, reaction mixture was cooled to room temperature, quenched with 5% aqueous sodium bicarbonate and extracted with ethyl acetate. The organic layer was washed with water and concentrated to yield crude title compound as brown coloured oil.

### Step (iii)

### Preparation of isopropyl 2(S)-trimethyl silyloxy-3-(4-hydroxyphenyl)propanoate (I)

In 250 ml parr hydrogenation flask, palladium carbon (5%, 1 g) slurred in water (1 ml) was taken. Isopropyl 2(S)-trimethyl silyloxy-3-(4-benzyloxyphenyl)propanoate (3 g) dissolved in isopropanol (100 ml) was added and hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 90-91%.

### EXAMPLE 22

### Step (i)

### Preparation of isopropyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) isopropanol (30 ml) and methane sulfonic acid (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 88-92%.

### Step (ii)

### Preparation of isopropyl 2(S)-trimethyl silyloxy-3-(4-benzyloxyphenyl)propanoate (VI)

In a 100 ml round bottom flask isopropyl 2(S)-hydroxy-3-(4-benzyloxyphenyl)propanoate (2.0 g), N-methyl pyrrolidone (20 ml), triethyl amine (1.60 g) were taken. Trimethyl silyl chloride (3.43 g, 0.0317 M) was added slowly. The reaction mass was heated to 60-70° C. and maintained at this temperature for a period of 20-24 h. The progress of the reaction was monitored by TLC. After completion of the reaction, the reaction mixture was cooled to room temperature, quenched with 5% aqueous sodium bicarbonate and extracted with ethyl acetate. The organic layer was washed with water and concentrated to yield crude title compound as brown coloured oil.

### Step (iii)

### Preparation of isopropyl 2(S)-trimethyl silyloxy-3-(4-hydroxyphenyl)propanoate (I)

In 250 ml parr hydrogenation flask, palladium carbon (5%, 1 g) slurred in water (1 ml) was taken. Isopropyl 2(S)-trimethyl silyloxy-3-(4-benzyloxyphenyl)propanoate (3 g) dissolved in THF (100 ml) was added and hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 79-83%.

### EXAMPLE 23

### Step (i)

### Preparation of isopropyl 2(S)-hydroxy-3-(4-benzyloxyphenyl)propanoate (V)

In a 50 ml 3 neck round bottom flask, fitted with a mechanical stirrer and reflux condenser 3-(4-benzyloxyphenyl)-2-hydroxypropanoic acid (3 g) isopropanol (30 ml) and thionyl chloride (0.3 ml) were taken and refluxed for 16 h. The progress of the reaction was monitored by TLC. Refluxing was continued, till the starting material has disappeared on TLC. The reaction mass was cooled to room temperature and transferred into a distillation flask and concentrated on a rotavapour. The concentrated mixture was diluted with ethyl acetate (30 ml), neutralized with saturated aqueous sodium bicarbonate solution. The organic layer was separated and the aqueous layer was extracted with ethyl acetate (30 ml). The combined organic layers were washed with water (30 ml) and concentrated on a rotavapour under reduced pressure to yield the title compound, yield 88-89%.

### Step (ii)

### Preparation of isopropyl 2(S)-trimethyl silyloxy-3-(4-benzyloxyphenyl)propanoate (VI)

In a 100 ml round bottom flask isopropyl 2(S)-hydroxy-3-(4-benzyloxyphenyl)propanoate (2.0 g), diethyl acetamide (20 ml), potassium carbonate (2.19 g) were taken. Trimethyl silyl chloride (3.43 g, 0.0317 M) was added slowly. The reaction mass was heated to 60-70° C. and maintained at this temperature for a period of 20-24 h. The progress of the reaction was monitored by TLC. After completion of the reaction, the reaction mixture was cooled to room temperature, quenched with 5% aqueous sodium bicarbonate and extracted with ethyl acetate. The organic layer was washed with water and concentrated to yield crude title compound as brown coloured oil.

### Step (iii)

### Preparation of isopropyl 2(S)-trimethyl silyloxy-3-(4-hydroxyphenyl)propanoate (I)

In 250 ml parr hydrogenation flask, palladium carbon (5%, 1 g) slurred in water (1 ml) was taken. Isopropyl 2(S)-trimethyl silyloxy-3-(4-benzyloxyphenyl)propanoate (3 g) dissolved in acetone (100 ml) was added and hydrogenated at 50-60 psi hydrogen pressure for 10-12 h. The reaction was monitored by TLC. After completion of the reaction, catalyst was filtered on a hi-flow bed and the solvent was evaporated on a rotavapour under reduced pressure to yield the title compound as a syrupy liquid, yield 78-84%.

### Demonstration of Efficacy of Compounds

Efficacy in Genetic Models

Mutation in colonies of laboratory animals and different sensitivities to dietary regimens have made the development of animal models with non-insulin dependent diabetes and hyperlipidemia associated with obesity and insulin resistance possible. Genetic models such as db/db and ob/ob (Diabetes, (1982) 31(1): 1-6) mice and zucker fa/fa rats have been developed by the various laboratories for understanding the pathophysiology of disease and testing the efficacy of new antidiabetic compounds (Diabetes, (1983) 32: 830-838; Annu. Rep. Sankyo Res. Lab. (1994). 46: 1-57). The homozygous animals, C57 BL/KsJ-db/db mice developed by Jackson Laboratory, US, are obese, hyperglycemic, hyperinsulinemic and insulin resistant (J. Clin. Invest., (1990) 85: 962-967), whereas heterozygous are lean and normoglycemic. In db/db model, mouse progressively develops insulinopenia with age, a feature commonly observed in late stages of human type II diabetes when blood sugar levels are insufficiently controlled. The state of pancreas and its course vary according to the models. Since this model resembles that of type II diabetes mellitus, the compounds of the present invention were tested for blood sugar and triglycerides lowering activities.

Male C57BL/KsJ-db/db mice of 8 to 14 weeks age, having body weight range of 35 to 60 grams, bred at Dr. Reddy's Research Foundation (DRF) animal house, were used in the experiment. The mice were provided with standard feed (National Institute of Nutrition (NIN), Hyderabad, India) and acidified water, ad libitum. The animals having more than 350 mg/dl blood sugar were used for testing. The number of animals in each group was 4.

Test compounds were suspended on 0.25% carboxymethyl cellulose and administered to test group at a dose of 0.1 mg to 30 mg/kg through oral gavage daily for 6 days. The control group received vehicle (dose 10 ml/kg). On 6th day the blood samples were collected one hour after administration of test compounds/vehicle for assessing the biological activity.

The random blood sugar and triglyceride levels were measured by collecting blood (100 μl) through orbital sinus, using heparinised capillary in tubes containing EDTA which was centrifuged to obtain plasma. The plasma glucose and triglyceride levels were measured spectrometrically, by glucose oxidase and glycerol-3-PO₄ oxidase/peroxidase enzyme (Dr. Reddy's Lab. Diagnostic Division Kits, Hyderabad, India) methods respectively.

The blood sugar and triglycerides lowering activities of the test compound was calculated according to the formula.

Formulae for Calculation:

Percent reduction in Blood sugar can be calculated according to the formula:

$$Percent reduction ( \% ) = [ 1 - \frac{TT / OT}{TC / OC} ] \times 100$$

OC=Zero day control group value

OT=Zero day treated group value

TC=Test day control group value

TT=Test day treated group value.

1. A compound of formula (I) 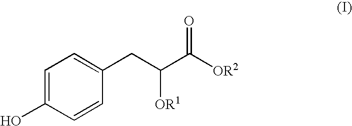
its derivatives, its stereoisomers, or its polymorphs wherein R¹ represents t-butyldimethyl silyl, trimethyl silyl or alkoxyalkyl group; R² represents hydrogen or substituted or unsubstituted (C₁-C₆) alkyl group.

2. A compound according to claim 1, wherein a substituent on the group represented by R² is selected from hydroxy or alkoxy group.

3. A compound according to claim 1, which is selected from the group consisting of:
(±) 3-(4-Hydroxyphenyl)-2-(t-butyl dimethyl silyloxy)propanoic acid;
(+) 3-(4-Hydroxyphenyl)-2-(t-butyl dimethyl silyloxy)propanoic acid;
(−) 3-(4-Hydroxyphenyl)-2-(t-butyl dimethyl silyloxy)propanoic acid;
(±) Methyl 3-(4-hydroxyphenyl)-2-(t-butyl dimethyl silyloxy)propanoate;
(+) Methyl 3-(4-hydroxyphenyl)-2-(t-butyl dimethyl silyloxy)propanoate;
(−) Methyl 3-(4-hydroxyphenyl)-2-(t-butyl dimethyl silyloxy) propanoate;
(±) Ethyl 3-(4-hydroxyphenyl)-2-(t-butyl dimethyl silyloxy)propanoate;
(+) Ethyl 3-(4-hydroxyphenyl)-2-(t-butyl dimethyl silyloxy)propanoate;
(−) Ethyl 3-(4-hydroxyphenyl)-2-(t-butyl dimethyl silyloxy)propanoate;
(±) Isopropyl 3-(4-hydroxyphenyl)-2-(t butyl dimethyl silyloxy)propanoate;
(+) Isopropyl 3-(4-hydroxyphenyl)-2-(t butyl dimethyl silyloxy)propanoate;
(−) Isopropyl 3-(4-hydroxyphenyl)-2-(t-butyl dimethyl silyloxy)propanoate;
(±) Isopropyl 3-(4-hydroxyphenyl)-2-methoxymethoxy propanoate;
(+) Isopropyl 3-(4-hydroxyphenyl)-2-methoxymethoxy propanoate;
(−) Isopropyl 3-(4-hydroxyphenyl)-2-methoxymethoxy propanoate;
(±) Isopropyl 3-(4-hydroxyphenyl)-2-methoxyethoxy propanoate;
(+) Isopropyl 3-(4-hydroxyphenyl)-2-methoxyethoxy propanoate;
(−) Isopropyl 3-(4-hydroxyphenyl)-2-methoxyethoxy propanoate;
(±) 3-(4-Hydroxyphenyl)-2-(trimethyl silyloxy)propanoic acid;
(+) 3-(4-Hydroxyphenyl)-2-(trimethyl silyloxy)propanoic acid;
(−) 3-(4-Hydroxyphenyl)-2-(trimethyl silyloxy)propanoic acid;
(±) Methyl 3-(4-hydroxyphenyl)-2-(trimethyl silyloxy)propanoate;
(+) Methyl 3-(4 hydroxyphenyl)-2-(trimethyl silyloxy)propanoate;
(−) Methyl 3-(4-hydroxyphenyl)-2-(trimethyl silyloxy)propanoate;
(±) Ethyl 3-(4-hydroxyphenyl)-2-(trimethyl silyloxy)propanoate;
(+) Ethyl 3-(4-hydroxyphenyl)-2-(trimethyl silyloxy)propanoate;
(−) Ethyl 3-(4-hydroxyphenyl)-2-(trimethyl silyloxy)propanoate;
(±) Isopropyl 3-(4-hydroxyphenyl)-2-(trimethyl silyloxy)propanoate;
(+) Isopropyl 3-(4-hydroxyphenyl)-2-(trimethyl silyloxy)propanoate; and
(−) Isopropyl 3-(4-hydroxyphenyl)-2-(trimethyl silyloxy)propanoate.

4. A process for the preparation of a compound of formula (I) 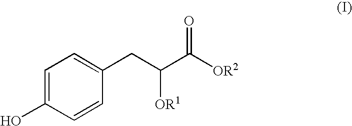
wherein R¹ represents t-butyldimethyl silyl, trimethyl silyl or alkoxyalkyl group; R² represents hydrogen or substituted or unsubstituted (C₁-C₆ ) alkyl group, which comprises the steps of:
(i) esterifying the compound of formula (IV) 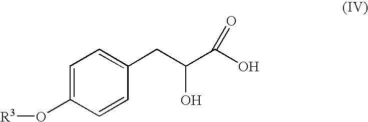
where R³ represents benzyl using an alkylating agent to produce a compound of formula (V) 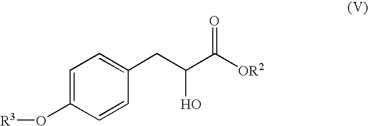
where R² represents (C₁-C₆)alkyl group,
ii) protecting the compound of formula (V) with a protecting agent in the presence of a base and a solvent to obtain compound of formula (VI) 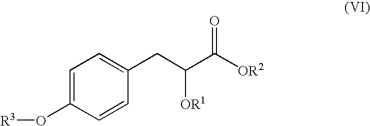
where R² represents a (C₁-C₆)alkyl group and R¹ and R³ are as defined above and
iii) debenzylating the compound of formula (VI) where R³ represents benzyl using aqueous alcohol in the presence of a metal catalyst to yield pure compound of formula (I) where R¹ and R² are as defined above.

5. The process as claimed in claim 4, wherein the esterification in step (I) is carried out using an alcohol under acidic conditions in the presence of sulfuric acid, methane sulfonic acid, thionyl chloride, amberlite resin or hydrochloric acid.

6. The process as claimed in claim 4, wherein the esterification in step (i) is carried out using ethyl iodide, DES or DMS under basic conditions in the presence of sodium carbonate, potassium carbonate or sodium methoxide.

7. The process as claimed in claim 4 wherein the esterification in step (i) is carried out at a temperature in the range of 30° C. to reflux temperature of the solvent used and the duration of the reaction is in the range from 2 to 20 h.

8. The process as claimed in claim 4, wherein the protection in step (ii) is carried out with a protecting agent selected from the group consisting of t-butyldimethyl silyl chloride, trimethyl silyl chloride and alkoxyalcohols.

9. The process as claimed in claim 4, wherein the protection in step (ii) is carried out in the presence of a base selected from the group consisting of imidazole, triethyl amine and potassium carbonate.

10. The process as claimed in claim 4, wherein the protection in step (ii) is carried out in the presence of a solvent selected from the group consisting of toluene, DMF, DCE, DCM, diethyl acetamide, methylpyrrolidone, ethyl acetate and acetonitrile.

11. The process as claimed in claim 4, wherein the protection in step (ii) is carried out at a temperature in the range of 10 to 90° C. and the duration of the reaction is in the range from 2-30 h.

12. The process as claimed in claim 4, wherein the debenzylation in step (iii) is carried out using THF, aqueous acetic acid, ethyl acetate or an aqueous (C₁-C₆) alcohol.

13. The process as claimed in claim 4, wherein the debenzylation in step (iii) is carried out in the presence of a metal catalyst.

14. An intermediate of formula (VI) 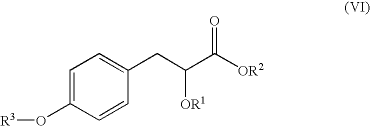
where R¹ represents t-butyldimethyl silyl, or trimethyl silyl or (C₁-C₆) alkyl group, R² represents hydrogen or (C₁-C₆ )alkyl group, and R³ represents benzyl.

15. A compound according to claim 14, which is selected from the group consisting of:
(±) 3-(4-Benzyloxyphenyl)-2-(t-butyl dimethyl silyloxy)propanoic acid;
(+) 3-(4-Benzyloxyphenyl)-2-(t-butyl dimethyl silyloxy)propanoic acid;
(−) 3-(4-Benzyloxyphenyl)-2-(t-butyl dimethyl silyloxy)propanoic acid;
(±) Methyl 3-(4-benzyloxyphenyl)-2-(t-butyl dimethyl silyloxy)propanoate,
(+) Methyl 3-(4-benzyloxyphenyl)-2-(t-butyl dimethyl silyloxy)propanoate;
(−) Methyl 3-(4-benzyloxyphenyl)-2-(t-butyl dimethyl silyloxy)propanoate,
(±) Ethyl 3-(4-benzyloxyphenyl)-2-(t-butyl dimethyl silyloxy)propanoate;
(+) Ethyl 3-(4-benzyloxyphenyl)-2-(t-butyl dimethyl silyloxy)propanoate;
(−) Ethyl 3-(4-benzyloxyphenyl)-2-(t-butyl dimethyl silyloxy)propanoate;
(±) Isopropyl 3-(4-benzyloxyphenyl)-2-(t-butyl dimethyl silyloxy)propanoate;
(+) Isopropyl 3-(4-benzyloxyphenyl)-2-(t-butyl dimethyl silyloxy)propanoate;
(−) Isopropyl 3-(4-benzyloxyphenyl)-2-(t-butyl dimethyl silyloxy)propanoate;
(±) 3-(4-Benzyloxyphenyl)-2-(trimethyl silyloxy)propanoic acid;
(+) 3-(4-Benzyloxyphenyl)-2-(trimethyl silyloxy)propanoic acid;
(−) 3-(4-Benzyloxyphenyl)-2-(trimethyl silyloxy)propanoic acid;
(±) Methyl 3-(4-hydroxyphenyl)-2-(trimethyl silyloxy)propanoate;
(+) Methyl 3-(4-hydroxyphenyl)-2-(trimethyl silyloxy)propanoate;
(−) Methyl 3-(4-hydroxyphenyl)-2-(t-butyl dimethyl silyloxy)propanoate;
(±) Ethyl 3-(4-hydroxyphenyl)-2-(trimethyl silyloxy)propanoate;
(+) Ethyl 3-(4-hydroxyphenyl)-2-(trimethyl silyloxy)propanoate;
(−) Ethyl 3-(4-hydroxyphenyl)-2-(trimethyl silyloxy)propanoate;
(±) Isopropyl 3-(4-benzyloxyphenyl)-2-(trimethyl silyloxy)propanoate;
(+) Isopropyl 3-(4-benzyloxyphenyl)-2-(trimethyl silyloxy)propanoate; and
(−) Isopropyl 3-(4-benzyloxyphenyl)-2-(trimethyl silyloxy)propanoate.

16 and 17 cancel.

18. The process as claimed in claim 5, wherein the alcohol is selected from the group consisting of methanol, ethanol, propanol or isopropanol.

19. The process as claimed in claim 5, wherein the esterification in step (i) is carried out using ethyl iodide, DES or DMS under basic conditions in the presence of sodium carbonate, potassium carbonate or sodium methoxide.

20. The process as claimed in claim 5, wherein the esterification in step (i) is carried out at a temperature in the range of 30° C. to reflux temperature of the solvent used and the duration of the reaction is in the range from 2 to 20 h.

21. The process as claimed in claim 6, wherein the esterification in step (i) is carried out at a temperature in the range of 30° C. to reflux temperature of the solvent used and the duration of the reaction is in the range from 2 to 20 h.

22. The process as claimed in claim 5, wherein the protection in step (ii) is carried out with a protecting agent selected from the group consisting of t-butyldimethyl silyl chloride, trimethyl silyl chloride and alkoxyalcohols.

23. The process as claimed in claim 6, wherein the protection in step (ii) is carried out with a protecting agent selected from the group consisting of t-butyldimethyl silyl chloride, trimethyl silyl chloride and alkoxyalcohols.

24. The process as claimed in claim 7, wherein the protection in step (ii) is carried out with a protecting agent selected from the group consisting of t-butyldimethyl silyl chloride, trimethyl silyl chloride and alkoxyalcohols.

25. The process according to claim 8, wherein the alkoxyalcohol is methoxymetanl or ethoxymethanol.

26. The process according to claim 22, wherein the alkoxyalcohol is methoxymetanl or ethoxymethanol.

27. The process as claimed in claim 5, wherein the protection in step (ii) is carried out in the presence of a base selected from the group consisting of imidazole, triethyl amine and potassium carbonate.

28. The process as claimed in claim 6, wherein the protection in step (ii) is carried out in the presence of a base selected from the group consisting of imidazole, triethyl amine and potassium carbonate.

29. The process as claimed in claim 7, wherein the protection in step (ii) is carried out in the presence of a base selected from the group consisting of imidazole, triethyl amine and potassium carbonate.

30. The process as claimed in claim 8, wherein the protection in step (ii) is carried out in the presence of a base selected from the group consisting of imidazole, triethyl amine and potassium carbonate.

31. The process as claimed in claim 5, wherein the protection in step (ii) is carried out in the presence of a solvent selected from the group consisting of toluene, DMF, DCE, DCM, diethyl acetamide, methylpyrrolidone, ethyl acetate and acetonitrile.

32. The process as claimed in claim 6, wherein the protection in step (ii) is carried out in the presence of a solvent selected from the group consisting of toluene, DMF, DCE, DCM, diethyl acetamide, methylpyrrolidone, ethyl acetate and acetonitrile.

33. The process as claimed in claim 7, wherein the protection in step (ii) is carried out in the presence of a solvent selected from the group consisting of toluene, DMF, DCE, DCM, diethyl acetamide, methylpyrrolidone, ethyl acetate and acetonitrile.

34. The process as claimed in claim 8, wherein the protection in step (ii) is carried out in the presence of a solvent solvents such as selected from the group consisting of toluene, DMF, DCE, DCM, diethyl acetamide, methylpyrrolidone, ethyl acetate and acetonitrile.

35. The process as claimed in claim 9, wherein the protection in step (ii) is carried out in the presence of a solvent solvents such as selected from the group consisting of toluene, DMF, DCE, DCM, diethyl acetamide, methylpyrrolidone, ethyl acetate and acetonitrile.

36. The process as claimed in claim 5, wherein the protection in step (ii) is carried out at a temperature in the range of 10 to 90° C. and the duration of the reaction is in the range from 2-30 h.

37. The process as claimed in claim 6, wherein the protection in step (ii) is carried out at a temperature in the range of 10 to 90° C. and the duration of the reaction is in the range from 2-30 h.

38. The process as claimed in claim 7, wherein the protection in step (ii) is carried out at a temperature in the range of 10 to 90° C. and the duration of the reaction is in the range from 2-30 h.

39. The process as claimed in claim 8, wherein the protection in step (ii) is carried out at a temperature in the range of 10 to 90° C. and the duration of the reaction is in the range from 2-30 h.

40. The process as claimed in claim 9, wherein the protection in step (ii) is carried out at a temperature in the range of 10 to 90° C. and the duration of the reaction is in the range from 2-30 h.

41. The process as claimed in claim 10, wherein the protection in step (ii) is carried out at a temperature in the range of 10 to 90° C. and the duration of the reaction is in the range from 2-30 h.

42. The process as claimed in claim 5, wherein the debenzylation in step (iii) is carried out using THF, aqueous acetic acid, ethyl acetate or an aqueous (C₁-C₆) alcohol.

43. The process as claimed in claim 12, wherein the alcohol is selected from the group consisting of methanol, ethanol or propanol.

44. The process as claimed in claim 42, wherein the alcohol is selected from the group consisting of methanol, ethanol or propanol.

45. The process as claimed in claim 5, wherein the debenzylation in step (iii) is carried out in the presence of a metal catalyst.

46. The process as claimed in claim 13, wherein the metal catalyst is Pd/C.

47. The process as claimed in claim 45, wherein the metal catalyst is Pd/C.

48. A method for treating diabetes, obesity, glucose intolerance, insulin resistance or disorders related to diabetes, obesity, glucose intolerance or insulin resistance selected from hypertension, coronary heart disease, atherosclerosis, stroke or peripheral vascular diseases, comprising administering an effective amount of a compound of formula (I) as defined in claim 1 to a patient in need thereof.

49. A method for treating diabetes, obesity, glucose intolerance, insulin resistance or disorders related to diabetes, obesity, glucose intolerance or insulin resistance selected from hypertension, coronary heart disease, atherosclerosis, stroke, or peripheral vascular diseases comprising administering an effective amount of a compound of claim 3 to a patient in need thereof.

50. A method for reducing total cholesterol, body weight, blood plasma glucose, triglycerides, LDL, VLDL or free fatty acids comprising administering an effective amount of a compound of formula (I) as defined in claim 1 to a patient in need thereof.

51. A method for reducing total cholesterol, body weight, blood plasma glucose, triglycerides, LDL, VLDL or free fatty acids comprising administering an effective amount of a compound as defined in claim 3 to a patient in need thereof.
